(19) United States
(12) Patent Application Publication
(10) Pub. No.: US 2023/0149298 A1
(43) Pub. Date: May 18, 2023

(54) Implantable Device for Treating an Inflammatory Eye Condition

(71) Applicant: Celanese EVA Performance Polymers LLC, Irving, TX (US)

(72) Inventors: Harsh Patel, Union, KY (US); Brian Wilson, Deerfield, IL (US); Jeffrey C. Haley, Norwood, OH (US)

(21) Appl. No.: 17/982,556

(22) Filed: Nov. 8, 2022

Related U.S. Application Data
(60) Provisional application No. 63/330,087, filed on Apr. 12, 2022.
(60) Provisional application No. 63/302,278, filed on Jan. 24, 2022.
(60) Provisional application No. 63/289,883, filed on Dec. 15, 2021.
(60) Provisional application No. 63/280,359, filed on Nov. 17, 2021.

(51) Int. Cl. A61K 9/00; A61K 31/573; A61K 47/32

(52) U.S. Cl. CPC A61K 9/0024; A61K 31/573; A61K 9/0048; A61K 47/32

(57) ABSTRACT

An implantable device for prohibiting and/or treating an inflammatory eye condition is provided. The device comprises a core defining an outer peripheral surface. The core comprises a core polymer matrix within which is dispersed at least a steroidal agent, the polymer matrix containing an ethylene vinyl acetate copolymer.

### RELATED APPLICATIONS

The present application is based upon and claims priority to U.S. Provisional Patent Application Ser. No. 63/280,359, having a filing date of Nov. 17, 2021; U.S. Provisional Patent Application Ser. No. 63/289,883, having a filing date of Dec. 15, 2021; U.S. Provisional Patent Application Ser. No. 63/302,278, having a filing date of Jan. 24, 2022; and U.S. Provisional Patent Application Ser. No. 63/330,087, having a filing date of Apr. 12, 2022, which are incorporated herein by reference.

### BACKGROUND

Several eye conditions are associated with inflammation. Ocular inflammation may lead to uveitis, age-related macular degeneration (AMD), diabetic retinopathy, among other conditions, and may result in vision reduction or permanent loss of vision. During ocular inflammation, macrophages (e.g., microglia) are activated and produce various pro-inflammatory cytokines, reactive oxygen species, growth factors, complement proteins, etc. resulting in inflammation. The development of AMD is associated with a process called choroidal neovascularization (CNV). Leakage from the CNV causes macular edema and collection of fluid beneath the macula resulting in vision loss. Diabetic macular edema (DME) is another eye disorder associated with ocular inflammation. DME is the most prevalent cause of moderate vision loss in patients with diabetes and is a common complication of diabetic retinopathy, a disease affecting the blood vessels of the retina. Clinically significant DME occurs when fluid leaks into the center of the macula, the light-sensitive part of the retina responsible for sharp, direct vision. Fluid in the macula can cause severe vision loss or blindness.

It is generally well known that certain steroidal agents (e.g., corticosteroids) can act as anti-inflammatory agents to help treat inflammation in the eye. For example, corticosteroids can effectively treat some forms of neovascularization such as corneal neovascularization. Unfortunately, when these compounds are used to treat neovascularization of the posterior segment by direct injection, they can cause undesirable side effects in many patients. The adverse effects or undesirable side effects being observed included elevations in intraocular pressure and the formation of, or acceleration of, the development of cataracts. Elevations in intraocular pressure are of particular concern in patients who are already suffering from elevated intraocular pressure, such as glaucoma patients. Moreover, a risk exists that the use of corticosteroids in patients with normal intraocular pressure will cause elevations in pressure that result in damage to ocular tissue. Since therapy with corticosteroids is frequently long term, i.e., several days or more, a potential exists for significant damage to ocular tissue as a result of prolonged elevations in intraocular pressure attributable to that therapy.

As such, a need continues to exist for an improved technique for the intraocular delivery of a steroidal agent for the treatment of inflammatory eye conditions in a patient.

### SUMMARY OF THE INVENTION

In accordance with one embodiment of the present invention, an implantable device for prohibiting and/or treating an inflammatory eye condition in a patient is disclosed. The device comprises a core that defines an outer peripheral surface, wherein the core comprises a core polymer matrix within which is dispersed a steroidal agent. The core polymer matrix contains an ethylene vinyl acetate copolymer.

Other features and aspects of the present invention are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of the implantable device of the present invention;

FIG. 2 is a cross-sectional view of the implantable device of FIG. 1;

FIG. 3 is a graph showing the cumulative release of dexamethasone per surface area versus time for Examples 1-4; and

FIG. 4 is a graph showing the total cumulative release of dexamethasone versus time for Examples 1-4.

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to an implantable device that is capable of intraocular delivery of a steroidal agent to a patient (e.g., human, pet, farm animal, racehorse, etc.) over a sustained period of time. The implantable device contains a core that defines an outer peripheral surface and an optional sheath that is disposed over at least a portion of the outer peripheral surface of the core. By selectively the controlling the particular materials used to form the core and/or sheath, as well as the particular manner in which they are constructed, the release rate of the steroidal agent can be controlled over an extended period of time. More particularly, the core contains one or more layers that include a steroidal agent dispersed within a core polymer matrix. The core polymer matrix, in turn, includes one or more ethylene vinyl acetate copolymers. The steroidal agent may be present in the core at a relative high loading, such as from about 20 wt. % to about 70 wt. %, in some embodiments from about 25 wt. % to about 65 wt. %, in some embodiments from about 30 wt. % to about 60 wt. %, and in some embodiments, from about 35 wt. % to about 55 wt. % of the core. In some embodiments, the steroidal agent comprises about 50 wt. % of the core. The polymer matrix may likewise constitute from about 30 wt. % to about 80 wt. %, in some embodiments from about 35 wt. % to about 80 wt. %, in some embodiments from about 40 wt. % to about 70 wt. %, and in some embodiments, from about 45 wt. % to about 65 wt. % of the core. When employed, the sheath may contain one or more layers that include a polymer matrix containing a hydrophobic polymer. In this manner, the sheath may be generally impermeable to the steroidal agent so that it is capable of being released primarily only through uncovered surfaces of the device.

Various embodiments of the present invention will now be described in more detail.

### I. Core

A. Polymer Matrix

As indicated above, the core polymer matrix contains at least ethylene vinyl acetate copolymer, which is generally derived from at least one ethylene monomer and at least one vinyl acetate monomer. Certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 55 wt. %, in some embodiments from about 30 wt. % to about 50 wt. %, in some embodiments from about 35 wt. % to about 48 wt. %, and in some embodiments, from about 38 wt. % to about 45 wt. % of the copolymer. Conversely, the ethylene content of the copolymer may likewise be within a range of from about 40 wt. % to about 80 wt. %, 45 wt. % to about 75 wt. %, in some embodiments from about 50 wt. % to about 80 wt. %, in some embodiments from about 52 wt. % to about 65 wt. %, and in some embodiments, from about 55 wt. % to about 62 wt. %. Among other things, such a comonomer content may help achieve a controllable, sustained release profile of the steroidal agent, while also still having a relatively low melting temperature that is more similar in nature to the melting temperature of the steroidal agent. The melt flow index of the ethylene vinyl acetate copolymer(s) and resulting polymer matrix may also range from about 0.2 to about 400 g/10 minutes, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. The melting temperature of the ethylene vinyl acetate copolymer may likewise be from about 20° C. to about 70° C., in some embodiments from about 25° C. to about 65° C., and in some embodiments, from about 30° C. to about 60° C., such as determined in accordance with ASTM D3418-15. Particularly suitable examples of ethylene vinyl acetate copolymers that may be employed include those available from Celanese under the designation ATEVA® (e.g., ATEVA® 4030AC), DuPont under the designation ELVAX® (e.g., ELVAX® 40W); and Arkema under the designation EVATANE® (e.g., EVATANE 40-55).

In certain embodiments, it may also be desirable to employ blends of an ethylene vinyl acetate copolymer and a hydrophobic polymer (as described in more detail below) such that the overall blend and polymer matrix have a melting temperature and/or melt flow index within the range noted above. For example, the core polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second ethylene copolymer may likewise constitute from about 20 wt. % to about 80 wt. %, in some embodiments from about 30 wt. % to about 70 wt. %, and in some embodiments, from about 40 wt. % to about 60 wt. % of the polymer matrix. Blends of an ethylene vinyl acetate copolymer and other types of hydrophobic polymers, such as described below, may also be employed.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain cases, ethylene vinyl acetate copolymer(s) constitute the entire polymer content of the polymer matrix. In other cases, however, it may be desired to include other polymers, such as other hydrophobic polymers and/or hydrophilic polymers as described in more detail below. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

If desired, the core polymer matrix may also contain one or more plasticizers to help lower the processing temperature, thereby allowing higher melting point copolymers to be used without degrading the steroidal agent. Suitable plasticizers may include, for instance, fatty acids, fatty acids esters, fatty acid salts, fatty acid amides, organic phosphate esters, hydrocarbon waxes, etc., as well as mixtures thereof. The fatty acid may generally be any saturated or unsaturated acid having a carbon chain length of from about 8 to 22 carbon atoms, and in some embodiments, from about 10 to about 18 carbon atoms. If desired, the acid may be substituted. Suitable fatty acids may include, for instance, lauric acid, myristic acid, behenic acid, oleic acid, palmitic acid, stearic acid, ricinoleic acid, capric acid, neodecanoic acid, hydrogenated tallow fatty acid, hydroxy stearic acid, the fatty acids of hydrogenated castor oil, erucic acid, coconut oil fatty acid, etc., as well as mixtures thereof. Fatty acid derivatives may also be employed, such as fatty acid amides, such as oleamide, erucamide, stearamide, ethylene bis(stearamide), etc.; fatty acid salts (e.g., metal salts), such as calcium stearate, zinc stearate, magnesium stearate, iron stearate, manganese stearate, nickel stearate, cobalt stearate, etc.; fatty acid esters, such as fatty acid esters of aliphatic alcohols (e.g., 2-ethylhexanol, monoethylene glycol, isotridecanol, propylene glycol, pentraerythritol, etc.), fatty acid esters of glycerols (e.g., castor oil, sesame oil, etc.), fatty acid esters of polyphenols, sugar fatty acid esters, etc.; as well as mixtures of any of the foregoing. Hydrocarbon waxes, including paraffin waxes, polyolefin and oxidized polyolefin waxes, and microcrystalline waxes, may also be employed. Particularly suitable are acids, salts, or amides of stearic acid, such as stearic acid, calcium stearate, pentaerythritol tetrastearate, or N,N′-ethylene-bis-stearamide. When employed, the plasticizer(s) typically constitute from about 0.05 wt. % to about 1.5 wt. %, and in some embodiments, from about 0.1 wt. % to about 0.5 wt. % of the polymer matrix.

B. Steroidal Agents

As noted above, one or more steroidal anti-inflammatory agents (herein “steroidal agent”) are dispersed within the core polymer matrix. The term “steroidal agent” generally refers to a molecule capable of reducing and/or treating inflammation. Such steroidal agents may comprise one or more corticosteroids, such as glucocorticoids. Glucocorticoids are defined as a subgroup of corticosteroids. Glucocorticoids, sometimes also named glucocorticosteroids, are a class of steroid hormones that bind to the glucocorticoid receptor and are part of the feedback mechanism of the immune system that turns down immune activity, (e.g., inflammation). In medicine, they are used to treat diseases that are caused by an overactive immune system, such as allergies, asthma, autoimmune diseases, and sepsis. They also interfere with some of the abnormal mechanisms in cancer cells, so that they are also used to treat cancer. Upon binding, the glucocorticoid receptor, the activated glucocorticoid receptor complex up-regulates the expression of anti-inflammatory proteins in the nucleus by a process known as transactivation and represses the expression of pro-inflammatory proteins in the cytosol by attenuating actions on gene induction (via NF-κB, AP1, jun-jun-homodimers, etc.).

Suitable examples of glucocorticoids may comprise hydrocortisone, cortisone acetate, cortisone/cortisol, fluorocortolone, fluocinolone, flourometholone, prednisone, prednisolone, methylprednisolone, triamcinolone, dexamethasone, betamethasone, paramethasone, etc., as well as derivatives and combinations thereof. Dexamethasone and derivatives thereof are particularly suitable. Glucocorticoid polymorphs, isomers, hydrates, solvates, or derivatives thereof are all meant to be encompassed in the scope of the present disclosure and shall be understood to fall under the term “glucocorticoid.”

The steroidal agent may be generally stable at high enough temperatures so that it can be incorporated into the polymer matrix at or near the melting temperature of the ethylene vinyl acetate polymer employed in the core without significantly degrading (e.g., melting) during manufacturing or use of the device. For example, the steroidal agent may remain stable at temperatures of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C. The steroidal agent may be inherently stable at such temperatures, or it may also be encapsulated or otherwise protected by a carrier component that is stable at such temperatures, such as a carrier component containing peptides, proteins, carbohydrates (e.g., sugars), polymers, lipids, etc. In one particular embodiment, for example, the carrier component may include a lipid, which generally refers to a small molecule that has hydrophobic or amphiphilic properties, such as fats, waxes, sterol-containing metabolites, vitamins, fatty acids, glycerolipids, glycerophospholipids, sphingolipids, saccharolipids, polyketides, and prenol lipids. Examples of such lipids may include, for instance, phospholipids, such as alkyl phosphocholines and/or fatty acid-modified phosphocholines (e.g., 1,2-dioleoyl-sn-glycero-3-phosphoethanolamine (DOPE) or 1,2-distearoyl-sn-glycero-3-phosphocholine (DSPC)); cationic lipids, such as 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)-butanoyl)oxy)heptadecanedioate (L319); helper lipids (e.g., fatty acids); structural lipids (e.g., sterols); polyethylene glycol (PEG)-conjugated lipids, etc., as well as combinations of any of the foregoing. Regardless, at least one of the compounds (e.g., lipid) employed in the carrier component may be selected to have a melting temperature that is similar to or higher than the melting temperature of the ethylene vinyl acetate copolymer(s) within the core. In fact, in certain embodiments, multiple compounds or even all of the compounds within the carrier component may be selected to have a melting temperature that is similar to or higher than the melting temperature of the ethylene vinyl acetate copolymer(s) within the polymer matrix. In this manner, the encapsulated steroidal agent can remain stable at or near the melt processing temperature of the ethylene vinyl acetate copolymer(s) employed in the core, which is generally higher than the melting temperature of such copolymer(s). For example, the ratio of the melting temperature (° C.) of the ethylene vinyl acetate copolymer(s) within the core to the melting temperature (° C.) of compound(s) (e.g., lipid(s)) within the carrier component may be about 2° C./° C. or less, in some embodiments about 1.8° C./° C. or less, in some embodiments from about 0.1 to about 1.6° C./° C., in some embodiments from about 0.2 to about 1.5° C./° C., and in some embodiments, from about 0.4 to about 1.2° C./° C. The ethylene vinyl acetate copolymer(s) and resulting polymer matrix may, for instance, have a melting temperature of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. The compound(s) within the carrier component (e.g., lipid(s) may likewise have a melting temperature of from about 25° C. to about 105° C., in some embodiments from about 30° C. to about 85° C., in some embodiments from about 35° C. to about 75° C., in some embodiments from about 40° C. to about 70° C., and in some embodiments, from about 45° C. to about 65° C.

C. Excipients

The core may also optionally contain one or more excipients, such as cell permeability enhancers, radiocontrast agents, hydrophilic compounds, bulking agents, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. The optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

To help further control the release rate from the implantable medical device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of a membrane layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted, or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

In certain cases, the hydrophilic compound may be in the form of water-soluble particles distributed within the core polymer matrix. The particle size of the water-soluble particles may be controlled to help achieve the desired delivery rate. More particularly, the median diameter (D50) of the particles may be about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles may also be selected to achieve the desired release profile. For example, the water-soluble particles may contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present invention include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof.

Regardless of the particular components employed, the core may be formed through a variety of known techniques, such as by hot-melt extrusion, injection molding, solvent casting, dip coating, spray coating, microextrusion, coacervation, etc. In one embodiment, a hot-melt extrusion technique may be employed. Hot-melt extrusion is generally a solvent-free process in which the components of the core (e.g., ethylene vinyl acetate copolymer(s), steroidal agent (s), optional excipients, etc.) may be melt blended and optionally shaped in a continuous manufacturing process to enable consistent output quality at high throughput rates. This technique is particularly well suited to ethylene vinyl acetate copolymers as they typically exhibit a relatively high degree of long-chain branching with a broad molecular weight distribution. This combination of traits can lead to shear thinning of the copolymer during the extrusion process, which help facilitates hot-melt extrusion. Furthermore, the polar vinyl acetate comonomer units can serve as an “internal” plasticizer by inhibiting crystallization of the polyethylene chain segments. This may lead to a lower melting point of the copolymer, which further enhances its ability to be processed with the steroidal agent.

During a hot-melt extrusion process, melt blending generally occurs at a temperature that is similar to or even less than the melting temperature of the steroidal agent or carrier component (e.g., lipid) for the steroidal agent. Melt blending may also occur at a temperature that is similar to or slightly above the melting temperature of the ethylene vinyl acetate copolymer(s). The ratio of the melt blending temperature to the melting temperature of the steroidal agent and/or carrier component therefor may, for instance, be about 2 or less, in some embodiments about 1.8 or less, in some embodiments from about 0.1 to about 1.6, in some embodiments from about 0.2 to about 1.5, and in some embodiments, from about 0.4 to about 1.2. The melt blending temperature may, for example, be from about 30° C. to about 100° C., in some embodiments, from about 40° C. to about 80° C., and in some embodiments, from about 50° C. to about 70° C. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical, and it may contain any number and/or orientation of threads and channels as is known in the art. For example, the screw typically contains a thread that forms a generally helical channel radially extending around the center of the screw. A feed section and melt section may be defined along the length of the screw. The feed section is the input portion of the barrel where the ethylene vinyl acetate copolymer(s) and/or steroidal agent are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, DuImage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be extruded through an orifice (e.g., die) and formed into pellets, sheets, fibers, filaments, etc., which may be thereafter shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present invention. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape for the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 50° C. to about 120° C., in some embodiments from about 60° C. to about 110° C., and in some embodiments, from about 70° C. to about 90° C.

As indicated above, another suitable technique for forming a core of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present invention. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

D. Non-Steroidal Therapeutic Agents

If desired, the implantable device may further comprise one or more non-steroidal therapeutic agents in combination with the steroidal agent. Examples of such non-steroidal therapeutic agents may include non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil); immunological drugs (such as vaccines and immune stimulants); hormonal agents (such as estrogens, estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotropin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, etc.

It may be particularly desired to employ a VEGF antagonist within the core polymer matrix in combination with a steroidal agent. The term “VEGF antagonist” generally refers to a molecule capable of neutralizing, blocking, inhibiting, abrogating, reducing, and/or interfering one or more biological activities (e.g., mitogenic, angiogenic and/or vascular permeability), including its binding to one or more VEGF receptors. Such antagonists may include anti-VEGF antibodies and antigen-binding fragments thereof, as well as non-antibody VEGF antagonists that include receptor molecules and derivatives that bind specifically to VEGF. Such antagonists are generally “macromolecular” in the sense that they have a large molecular weight, such as about 0.5 kilodaltons (“kDa”) or more, in some embodiments about 1 kDa or more, in some embodiments from about 5 kDa to about 250 kDa, and in some embodiments, from about 20 kDa to about 200 kDa.

Particularly suitable VEGF antagonists include anti-VEGF antibodies that bind to VEGF with sufficient affinity and specificity. The term “antibody” includes, by way of example, both naturally occurring and non-naturally occurring Abs, monoclonal and polyclonal Abs, chimeric and humanized Abs, human or nonhuman Abs, wholly synthetic Abs, single chain Abs, etc. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab. Particularly suitable antibodies may include monoclonal antibodies (“MAbs”), multispecific (e.g., bispecific) antibodies, or combinations thereof. The term “monoclonal antibody” generally refers to a non-naturally occurring preparation of Ab molecules of single molecular composition, i.e., Ab molecules whose primary sequences are essentially identical, and which exhibits a single binding specificity and affinity for a particular epitope. Multispecific antibodies, on the other hand, can bind simultaneously different antigens (e.g., two antigens). Such antibodies are generally produced by hybridoma, recombinant, transgenic or other techniques known to those skilled in the art. A “human” antibody refers to an Ab having variable regions in which both the framework and CDR regions are derived from human germline immunoglobulin sequences. Furthermore, if the Ab contains a constant region, the constant region also is derived from human germline immunoglobulin sequences. The human Abs may include amino acid residues not encoded by human germline immunoglobulin sequences (e.g., mutations introduced by random or site-specific mutagenesis in vitro or by somatic mutation in vivo). However, the term “human antibody”, as used herein, is not intended to include Abs in which CDR sequences derived from the germline of another mammalian species, such as a mouse, have been grafted onto human framework sequences.

The term “anti-VEGF antibody” refers to an antibody or antibody fragment, such as a Fab or a scFV fragment, that specifically binds to VEGF and inhibits one or more of its biological activities, e.g., its mitogenic, angiogenic and/or vascular permeability activity. Anti-VEGF antibodies act, for example, by interfering with the binding of VEGF to a cellular receptor, by interfering with vascular endothelial cell activation after VEGF binding to a cellular receptor, and/or by killing cells activated by VEGF. An anti-VEGF antibody will usually not bind to other VEGF homologues (e.g., VEGF-B or VEGF-C) or other growth factors (e.g., PIGF, PDGF or bFGF). Suitable anti-VEGF antibodies may include monoclonal and/or bispecific anti-VEGF antibodies, such as A4.6.1, bevacizumab, ranibizumab, G6, B20, 2C3, and other antibodies such as described in U.S. Pat. Nos. 6,582,959, 6,703,020, 7,060,269, 7,169,901, 7,691,977, and 10,590,193; U.S. Patent Publication No. 2009/0169556; WO 94/10202; WO 98/45332; WO 96/30046, WO 2019/154776; WO 2010/040508; WO 2011/117329; WO 2012/131078; WO 2015/083978; WO 2017/197199; and WO 2014/009465, all of which are incorporated herein by reference. For example, the anti-VEGF antibody may be ranibizumab, bevacizumab, or an antigen-binding fragment thereof. Ranibizumab (molecular weight of 48 kD) is a humanized monoclonal Fab fragment directed against VEGF-A having the light and heavy chain variable domain sequences of Y0317 as described in SEQ ID Nos. 115 and 116 of U.S. Pat. No. 7,060,269. Ranibizumab inhibits endothelial cell proliferation and neovascularisation and may be used for the treatment of neovascular (wet) age-related macular degeneration (AMD), the treatment of visual impairment due to diabetic macular oedema (DME), the treatment of visual impairment due to macular oedema secondary to retinal vein occlusion (branch RVO or central RVO), or treatment of visual impairment due to choroidal neovascularisation (CNV) secondary to pathologic myopia. Bevacizumab (molecular weight of 149 kD) is likewise a full-length, humanized murine monoclonal antibody that recognizes all isoforms of VEGF, and which is the parent antibody of ranibizumab. In another embodiment, the anti-VEGF antibody may also be a bispecific antibody that contains a first antigen binding site that binds to human vascular endothelial growth factor (e.g., VEGF-A) and a second antigen binding site that binds to human angiopoietin-2 (ANG-2). One example of such an anti-VEGF antibody is faricimab (molecular weight of 150 kD), which is described in WO 2019/154776 and WO 2014/009465 and is composed of an anti-Ang-2 antigen-binding fragment (Fab), an anti-VEGF-A Fab, and a modified fragment crystallizable region (Fc region).

As indicated, non-antibody VEGF antagonists may also be employed, such as VEGF traps, aptamers, and/or antibody mimetics such as described in WO 00/75319, WO 2010/060748, and WO 2011/135067, which are incorporated herein by reference. Specific examples of such non-antibody VEGF antagonists include aflibercept and pegaptanib. Aflibercept (molecular weight of 115 kD) is known as a VEGF-trap and is a recombinant human soluble VEGF receptor fusion protein in which portions of human VEGF receptors 1 and 2 extracellular domains are fused to the Fc portion of human IgG1. Pegaptanib (molecular weight of 50 kD) is a pegylated anti-vascular endothelial growth factor (VEGF) aptamer, a single strand of nucleic acid that binds with specificity to a particular target. Pegaptanib specifically binds to the 165 isoform of VEGF, a protein that plays a critical role in angiogenesis (the formation of new blood vessels) and increased permeability (leakage from blood vessels), two of the primary pathological processes responsible for the vision loss associated with neovascular AMD. Antibody mimetics that are VEGF antagonists may include, for instance, binding proteins containing an ankyrin repeat domain that binds VEGF and inhibits its binding to the receptor, such as DARPin® MP0112.

Similar to the steroidal agent, the non-steroidal therapeutic agent may also be generally stable at high enough temperatures so that it can be incorporated into the polymer matrix at or near the melting temperature of the ethylene vinyl acetate polymer employed in the core without significantly degrading (e.g., melting) during manufacturing or use of the device. For example, the VEGF antagonist may be inherently stable at temperatures as described above or otherwise protected by a carrier component that is stable at such temperatures, such as a carrier component containing peptides, proteins, carbohydrates (e.g., sugars), polymers, lipids, etc.

### II. Other Components

If desired, the implantable device may also contain various other components to help provide the desired release rate for the steroidal agent.

A. Membrane

In one embodiment, for example, a membrane may be employed that is disposed over at least a portion of the outer peripheral surface and/or one or more ends of the core. The membrane may cover the entire outer peripheral surface and two ends of the core or, in some cases, only the first edge and/or second edge of the device to help facilitate release of the steroidal agent. The number of membrane layers may vary depending on the particular configuration of the device, the nature of the steroidal agent, and the desired release profile. For example, the device may contain only one membrane layer.

When employed, the membrane layer(s) generally contains a plurality of water-soluble particles, such as described above, distributed within a membrane polymer matrix. The membrane polymer matrix may, for instance, contain at least one ethylene vinyl acetate copolymer, such as described in more detail above. In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments from about 60 wt. % to about 96 wt. %, and in some embodiments, from about 70 wt. % to about 95 wt. % of a membrane layer. Likewise, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix that includes a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different the copolymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a copolymer employed in the core. Among other things, this can further help control the release of the steroidal agent from the device. For example, the ratio of the melt flow index of a copolymer employed in the core to the melt flow index of an ethylene vinyl acetate copolymer employed in the membrane layer(s) may be from about 1 to about 20, in some embodiments about 2 to about 15, and in some embodiments, from about 4 to about 12.

If desired, membrane layer(s) used in the device may optionally contain a steroidal agent, such as described above, which is also dispersed within the polymer matrix. The steroidal agent in the membrane layer(s) may be the same or different than the steroidal agent employed in the core. When such a steroidal agent is employed in a membrane layer, the membrane layer generally contains the steroidal agent in an amount such that the ratio of the concentration (wt. %) of the steroidal agent in the core to the concentration (wt. %) of steroidal agent in the membrane layer is greater than 1, in some embodiments about 1.5 or more, and in some embodiments, from about 1.8 to about 4. When employed, steroidal agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of such steroidal agents prior to release from the core. When multiple membrane layers are employed, each membrane layer may generally contain the steroidal agent in an amount such that the ratio of the weight percentage of the steroidal agent in the core to the weight percentage of the steroidal agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

B. Sheath

The implantable medical device may also optionally include a sheath that is disposed over at least a portion of the outer peripheral surface of the core (including any optional membrane layer(s) disposed over the outer peripheral surface) so that the steroidal agent is capable of being released from the device primarily through uncovered surfaces.

To help control the release rate in this manner, the sheath may be formed from one or more layers that include a sheath polymer matrix containing a hydrophobic polymer. The sheath polymer matrix typically constitutes from about 60 wt. % to 100 wt. %, in some embodiments, from about 70 wt. % to 100 wt. %, and in some embodiments, from about 90 wt. % to 100 wt. % (e.g., 100 wt. %) of the sheath. The sheath is generally free of steroidal agents, but if present at all, they typically constitute less than about 10 wt. %, in some less than about 5 wt. %, and in some embodiments, from about 0.001 wt. % to about 4 wt. % of the sheath.

The sheath polymer matrix may be formed from a single hydrophobic polymer or blend of hydrophobic polymers. Examples of suitable hydrophobic polymers for use in the sheath polymer matrix may include, for instance, silicone polymers, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. In certain embodiments, the sheath polymer matrix may contain a semi-crystalline olefin copolymer. The melting temperature of such an olefin copolymer may, for instance, range from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth.

In one particular embodiment, the sheath polymer matrix may contain at least one ethylene vinyl acetate polymer. When employed, certain aspects of the copolymer can be selectively controlled to help ensure that the sheath remains generally impermeable to the steroidal agent. For instance, the vinyl acetate content of the copolymer may be selectively controlled to be within a range of from about 10 wt. % to about 50 wt. %, in some embodiments from about 15 wt. % to about 40 wt. %, in some embodiments from about 20 wt. % to about 35 wt. %, and in some embodiments, from about 24 wt. % to about 32 wt. % of the copolymer. In fact, in some cases, the vinyl acetate of the polymer content in the core polymer matrix may be greater than the vinyl acetate content in the sheath polymer matrix such that the ratio of the respective vinyl acetate contents is from about 1 to about 2.5, in some embodiments from about 1.2 to about 2, and in some embodiments, from about 1.4 to about 1.9. The ethylene content of the copolymer in the sheath polymer matrix may likewise be within a range of from about 50 wt. % to about 90 wt. %, in some embodiments from about 60 wt. % to about 85 wt. %, in some embodiments from about 65 wt. % to about 80 wt. %, and in some embodiments, from about 68 wt. % to about 76 wt. %. The melt flow index of the ethylene vinyl acetate copolymer(s) and resulting sheath polymer matrix may also range from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 2861A); Dow under the designation ELVAX® (e.g., ELVAX® 240W); and Arkema under the designation EVATANE® (e.g., EVATANE 28-40).

The sheath and any optional membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In one embodiment, a hot-melt extrusion technique may be employed. The core, membrane, and sheath may also be formed separately or simultaneously. In one embodiment, for instance, the core, membrane, and sheath are separately formed and then combined together using a known bonding technique, such as by stamping, hot sealing, adhesive bonding, etc. Compression molding (e.g., vacuum compression molding) may also be employed to form the implantable device. As described above, the core, membrane, and sheath may be each individually formed by heating and compressing the respective polymer compression into the desired shape while under vacuum. Once formed, the core, membrane, and sheath may be stacked together to form a multi-layer precursor and thereafter and compression molded in the manner as described above to form the resulting implantable device.

### III. Device Configuration

The implantable medical device may have a variety of different geometric shapes, such as cylindrical (rod), disc, ring, doughnut, helical, elliptical, triangular, ovular, etc. In one embodiment, for example, the implantable medical device may have a generally circular cross-sectional so that the overall structure is in the form of a cylinder (rod) or disc. The implantable medical device also has a relatively small size, such as a thickness (e.g., diameter) of from about 0.1 to about 10 millimeters, in some embodiments from about 0.1 to about 5 millimeters, in some embodiments from about 0.3 to about 2 millimeters, and in some embodiments, from about 0.4 to about 0.8 millimeters. The length of the implantable medical device may vary, but is typically from about 1 to about 250 millimeters, in some embodiments from about 2 to about 200 millimeters, in some embodiments from about 10 to about 150 millimeters, and in some embodiments, from about 20 to about 100 millimeters.

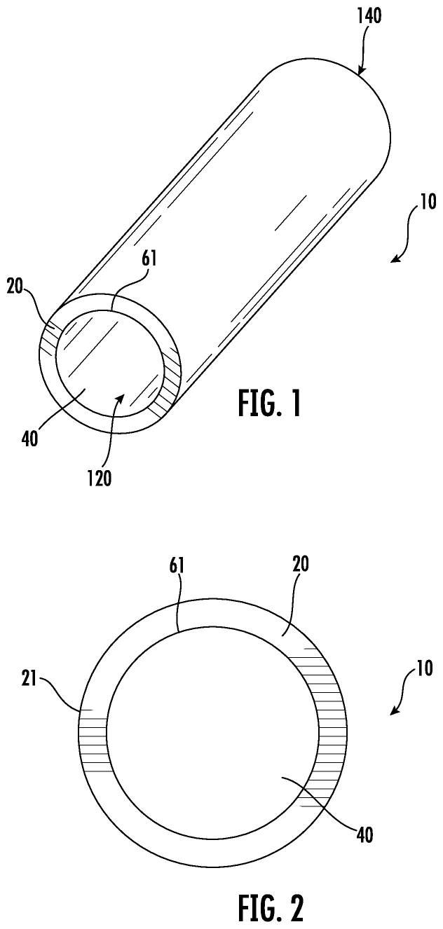

Regardless of its particular size or shape, the core of the device defines an outer peripheral surface, at least a portion of which is covered by the sheath. Referring to FIGS. 1-2, for example, one embodiment of an implantable medical device 10 is shown that contains a core 40 that defines an outer peripheral surface 61 extending between a first longitudinal edge 120 and a second longitudinal edge 140. A sheath 20 is circumferentially disposed over at least a portion of the surface 61. The sheath may, for instance, cover about 50% or more, in some embodiments about 70% or more, and in some embodiments, from about 80% to 100% of the outer peripheral surface. In the illustrated embodiment, for example, the sheath 20 covers the entire outer peripheral surface 61 so that the steroidal agent is not generally significantly released from an external peripheral surface 21 of the device. Instead, the first edge 120 and the second edge 140 in this embodiment are not covered by the sheath 20 so that the by steroidal agent is released primarily through the first edge 120 and second edge 140. By confining the primary release of the steroidal agent to a relatively small area, the release can be controlled over extended periods of time (e.g., days, months, etc.).

Although not depicted herein, various other possible embodiments and configurations are also contemplated. In one embodiment, for instance, the sheath 20 of FIGS. 1-2 may cover one edge of the device (e.g., first edge 120) so that the steroidal agent is primarily released through the other edge (e.g., second edge 140). Alternatively, the sheath 20 may cover at least a portion of both longitudinal edges 120 and 140, but be applied in a pattern such that the steroidal agent is capable of being released only through the uncovered areas of the edge(s). In yet other embodiments, the device may not contain any sheath 20. The device may be generally disc-shaped in nature in certain cases. In such embodiments, the core may define a circumferential edge that is defined between an upper outer peripheral surface and a lower outer peripheral surface. A first sheath layer may optionally be disposed over the upper surface and a second sheath layer may optionally be disposed over the lower surface. The first and second sheath layers may be the same or different but are generally formed from the materials described above. In this particular configuration, at least a portion of the circumferential edge (e.g., the entire edge) may remain uncovered by the sheath layers. Thus, during use, the steroidal agent is capable of being released from the core and exit from the circumferential edge of the device.

### IV. Use of Device

Through selective control over the particular nature of the core and sheath, the resulting device can be effective for sustained intraocular release of the steroidal agent over a prolonged period of time. For example, the implantable device can release the steroidal agent for a time period of about 5 days or more, in some embodiments about 20 days or more, in some embodiments about 30 days or more, in some embodiments about 60 days or more, in some embodiments about 90 days or more, and in some embodiments, from about 120 days to about 360 days (e.g., about 180 days). Further, the steroidal agent can be released in a controlled manner (e.g., zero order or near zero order) over the course of the release time period. After a time period of 30 days, for example, the cumulative release ratio of the implantable device may be from about 20% to about 70%, in some embodiments from about 30% to about 65%, and in some embodiments, from about 40% to about 60%. Likewise, after a time period of 60 days, the cumulative release ratio of the implantable device may still be from about 40% to about 85%, in some embodiments from about 50% to about 80%, and in some embodiments, from about 60% to about 80%. The “cumulative release ratio” may be determined by dividing the amount of the steroidal agent released at a particulate time interval by the total amount of steroidal agent initially present, and then multiplying this number by 100.

Notably, given that the steroidal agent can be released from an outer surface of the device or one or more of the end surfaces of the device. The device can be configured to have a cumulative release relative to the surface area or a portion of the surface area of the device. For instance, after a time period of about 0 to 5 days the device can have a cumulative release per surface area of from about 0.1 mg/cm² to about 0.2 mg/cm². In other embodiments, after a time period of about 5 days to about 10 days, the device can have a cumulative release per surface area of from about 0.1 mg/cm² to about 0.3 0.1 mg/cm². In some embodiments, after at time period of about 10 days to about 15 days, the device can exhibit a cumulative release per surface area of from about 0.25 mg/cm² to about 0.33 mg/cm². In some embodiments, after at time period of about 15 days to about 20 days, the device can exhibit a cumulative release per surface area of from about 0.33 mg/cm² to about 0.50 mg/cm². In some embodiments, after at time period of about 20 days to about 25 days, the device can exhibit a cumulative release per surface area of from about 0.50 mg/cm² to about 0.60 mg/cm². In some embodiments, after at time period of about 25 days to about 30 days, the device can exhibit a cumulative release per surface area of from about 0.50 mg/cm² to about 0.70 mg/cm². In certain embodiments, at a time period of about 30 days the device can exhibit a cumulative release per surface area of greater than 0.60 mg/cm², such as from about 0.60 mg/cm² to about 0.67 mg/cm².

Further, the device can be configured such that the steroidal agent can be released in different amounts, such as different cumulative amounts over time. For instance, in an embodiment, the device includes a core containing about 50 wt. % of a steroidal agent and about 50 wt. % of an ethylene vinyl acetate copolymer having a vinyl acetate content of about 40%. Such a device is capable of having a total cumulative release of steroidal agent of from about 0.2 mg to about 0.5 mg on day 1, about 0.5 mg to about 0.8 mg on day 2, about 0.8 mg to about 1.2 mg on day 7, about 1.0 mg to about 1.5 mg on day 14, about 1.5 mg to about 2 mg on day 21, and about 2.0 mg to about 2.5 mg on day 28. In another embodiment, the device includes a core containing about 20 wt. % of a steroidal agent and about 80 wt. % of an ethylene vinyl acetate copolymer having a vinyl acetate content of about 40%. Such a device is capable of having a total cumulative release of steroidal agent of from about 0.2 mg to about 0.5 mg on day 1, about 0.2 mg to about 0.5 mg on day 2, about 0.5 mg to about 0.8 mg on day 7, about 0.5 mg to about 1.0 mg on day 14, about 1.1 mg to about 1.7 mg on day 21, and about 1.5 mg to about 2.0 mg on day 28. In another embodiment, the device includes a core containing about 50 wt. % of a steroidal agent and about 50 wt. % of an ethylene vinyl acetate copolymer having a vinyl acetate content of about 28%. Such a device is capable of having a total cumulative release of steroidal agent of from about 0.2 mg to about 0.5 mg on day 1, about 0.5 mg to about 0.8 mg on day 2, about 0.8 mg to about 1.2 mg on day 7, about 1.0 mg to about 1.5 mg on day 14, about 1.5 mg to about 2 mg on day 21, and about 2.0 mg to about 2.5 mg on day 28. In still another embodiment, the device includes a core containing about 20 wt. % of a steroidal agent and about 80 wt. % of an ethylene vinyl acetate copolymer having a vinyl acetate content of about 28%. Such a device is capable of having a total cumulative release of steroidal agent of from about 0.2 mg to about 0.5 mg on day 1, about 0.3 mg to about 0.5 mg on day 2, about 0.5 mg to about 1.0 mg on day 7, about 0.5 mg to about 1.0 mg on day 14, about 0.5 mg to about 1.0 mg on day 21, and about 1.0 mg to about 1.5 mg on day 28.

Of course, the actual dosage level of the steroidal agent delivered will vary depending on the particular steroidal agent employed and the time period for which it is intended to be released. The dosage level is generally high enough to provide a therapeutically effective amount of the steroidal agent to render a desired therapeutic outcome, i.e., a level or amount effective to reduce or alleviate symptoms of the condition for which it is administered. More particularly, a used herein, the phrase “therapeutically effective amount” means a dose of the steroidal agent that results in a detectable improvement in one or more symptoms or indicia of an inflammatory eye condition, or a dose of steroidal agent that inhibits, prevents, lessens, or delays the progression of an inflammatory eye condition. The exact amount necessary will vary, depending on the subject being treated, the age and general condition of the subject to which the steroidal agent is to be delivered, the capacity of the subject's immune system, the degree of effect desired, the severity of the condition being treated, the particular steroidal agent selected and mode of administration of the composition, among other factors. In one embodiment, for example, a therapeutically effective amount can be from about 0.05 mg to about 5 mg, in some embodiments from about 0.1 mg to about 4 mg, and in some embodiments, from about 0.5 to about 3 mg. The amount of the steroidal agent contained within the individual doses may be expressed in terms of milligrams of drug per kilogram of patient body weight (i.e., mg/kg). For example, the steroidal agent may be administered to a patient at a dose of about 0.0001 to about 10 mg/kg of patient body weight. Further, in embodiments where the device is inserted directly into an affected tissue, the amount of drug necessary to be therapeutically effective may be lower than if the drug is administered via other routes. For instance, if the drug is administered via an oral route, typically a higher amount of drug is administered to account for first pass through the liver and other systemic degradation effect on the drug. However, as provided herein, when the drug is provided by the implant directly into affected tissues, the drug passes from the device directly into the affected tissue, thus, lower amounts of drugs may be therapeutically effective.

As noted above, the implantable device may be particularly suitable for treatment of an inflammatory eye condition, which includes any condition and/or disease of the eye which is caused by or associated with an infection or an autoimmune disease. Non-limiting examples of inflammatory eye conditions that are treatable using the methods of the present invention include choroiditis, episcleritis, sarcoidosis, scleritis, ocular cicatricial pemphigoid, orbital inflammatory syndrome (e.g., orbital myositis, orbital pseudotumor, etc.), uveitis, infection corneal ulcers, endophthalmitis, keratitis, conjunctivitis, thyroid eye disease, etc.

The manner in which the device of the present invention is implanted within the eye of a patient may vary as known to those skilled in the art. For example, the device may be inserted into the anterior segment (anterior chamber between the posterior surface of the cornea and the iris and/or the posterior chamber between the iris and front face of the vitreous humor), the posterior segment (e.g., anterior hyaloid membrane, vitreous humor, retina, choroid, etc.), or a combination thereof. When treating inflammatory eye conditions, such as uveitis, infection corneal ulcers, endophthalmitis, keratitis, conjunctivitis, and thyroid eye disease, it is generally desired that the therapeutic agent is delivered to the posterior segment. To avoid having to pass through the blood-aqueous barrier, the implantable device may be directly inserted into the posterior segment, such as into the vitreous humor (“intravitreally”). Intravitreal injection techniques are well known in the art any may include, for instance, the use of a hollow needle through which the implant is passed. The needle may have a small diameter size (e.g., 18 to 30 gauge needle) so that the incision is self-sealing and the implantation occurs in a closed chamber. A self-sealing incision may also be formed using a conventional “tunneling” procedure in which a spatula-shaped scalpel is used to create a generally inverted V-shaped incision through the cornea. In a preferred mode, the instrument used to form the incision through the cornea remains in place (that is, extends through the corneal incision) during the procedure and is not removed until after implantation. Such incision-forming instrument either may be used to place the ocular implant or may cooperate with a delivery instrument to allow implantation through the same incision without withdrawing the incision-forming instrument. Of course, in other modes, various surgical instruments may be passed through one or more corneal incisions multiple times.

The device may be implanted subcutaneously, orally, mucosally, etc., using standard techniques. The delivery route may be intrapulmonary, gastroenteral, subcutaneous, intramuscular, or for introduction into the central nervous system, intraperitoneum or for intraorgan delivery. Despite being particularly beneficial for treating and/or prohibiting an inflammatory eye condition, the implantable device may also be suitable for delivering a steroidal agent to treat other conditions, such as cancer, allergies, inflammation, immunologically-mediated diseases, metabolic diseases, etc. In such embodiments, the device may be placed in a tissue site of a patient in, on, adjacent to, or near a tumor, such as a tumor of the pancreas, binary system, gallbladder, liver, small bowel, colon, brain, lung, eye, etc. If desired, the device may also be employed together with current systemic chemotherapy, external radiation, and/or surgery.

If desired, the implantable device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### Examples 1-4

Ateva® 4030AC and 2825A were compounded with dexamethasone via a 11-mm twin-screw extruder. Two different loading percentages (20 wt. % and 50 wt. %) were selected for dexamethasone as shown in Table 1. In total, four different formulations were produced, and the diameter of the compounded filaments were 2 mm. The filaments were cut into rods having a length of 1 cm.

| | Diameter (2 mm) | |
|---|---|---|
| Example | Length (1 cm) | |
| | Ateva ® 4030AC (wt. %) | Dexamethasone (wt. %) |
| 1 | 50 | 50 |
| 2 | 80 | 20 |
| | Ateva ® 2825A (wt. %) | Dexamethasone (wt. %) |
| 3 | 50 | 50 |
| 4 | 80 | 20 |

Once formed, the release of dexamethasone from rods into PBS buffer was measured in a shaking incubator maintained at 37° C. At regular intervals, the buffer was exchanged with fresh buffer, and the removed buffer characterized using ultra performance liquid chromatography (UPLC) technique.

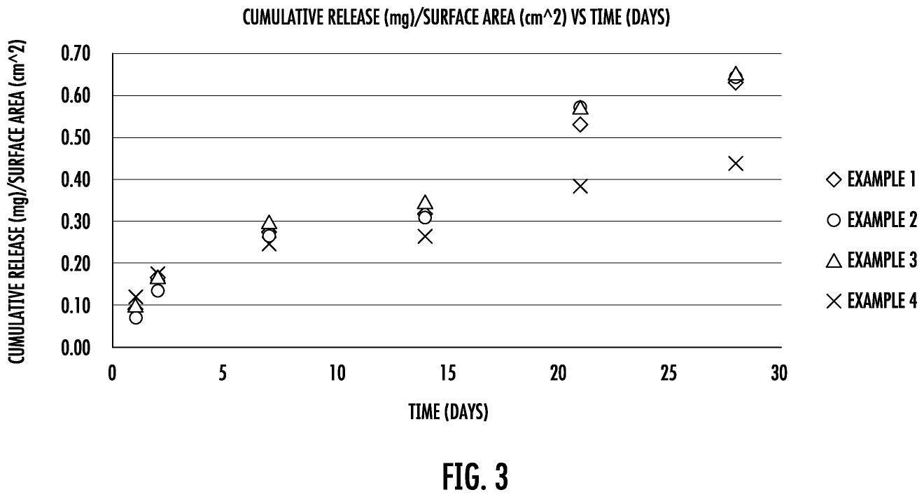

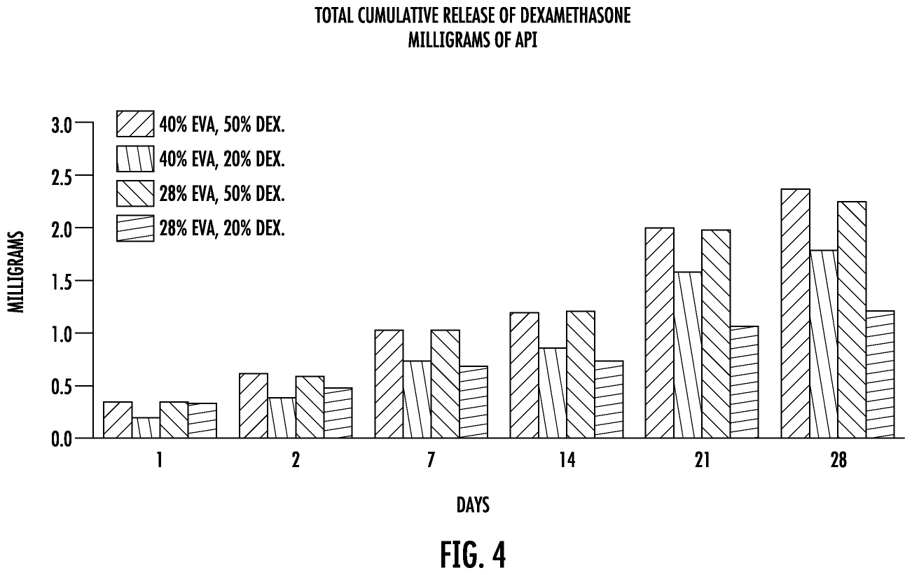

The release of dexamethasone per surface area and total cumulative release of dexamethasone from Examples 1-4 are provided in FIGS. 3 and 4, respectively.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An implantable device for prohibiting and/or treating an inflammatory eye condition in a patient, the device comprising a core that defines an outer peripheral surface, wherein the core comprises a core polymer matrix within which is dispersed a steroidal agent, the core polymer matrix containing an ethylene vinyl acetate copolymer.

2. The implantable device of claim 1, wherein the ethylene vinyl acetate copolymer has a vinyl acetate monomer content of from about 10 wt. % to about 60 wt. %, a melt flow index of from about 0.2 to about 100 grams per 10 minutes as determined in accordance with ASTM D1238-20 at a temperature of 190° C. and a load of 2.16 kilograms, and/or a melting temperature of from about 20° C. to about 70° C. as determined in accordance with ASTM D3418-15.

3. The implantable device of claim 1, wherein the steroidal agent constitutes from about 20 wt. % to about 70 wt. % of the core and the core polymer matrix is from about 30 wt. % to about 80 wt. %.

4. The implantable device of claim 1, wherein the steroidal agent comprises hydrocortisone, cortisone acetate, cortisone/cortisol, fluorocortolone, fluocinolone, fluorometholone, prednisone, prednisolone, methylprednisolone, triamcinolone, dexamethasone, betamethasone, paramethasone, derivatives thereof, or a combination of any of the foregoing.

5. The implantable device of claim 1, wherein the steroidal agent comprises dexamethasone or a derivative thereof.

6. The implantable device of claim 1, wherein a non-steroidal therapeutic agent is also dispersed within the core polymer matrix.

7. The implantable device of claim 6, wherein the non-steroidal therapeutic agent comprises a VEGF antagonist.

8. The implantable device of claim 1, wherein the core further comprises a hydrophilic compound.

9. The implantable device of claim 1, further comprising a membrane that is disposed over at least a portion of the outer peripheral surface.

10. The implantable device of claim 9, wherein the membrane contains a layer that includes a plurality of water-soluble particles dispersed within a membrane polymer matrix, wherein the membrane polymer matrix contains an ethylene vinyl acetate copolymer.

11. The implantable device of claim 1, wherein the device further comprises a sheath disposed over at least a portion of the outer peripheral surface, wherein the sheath comprises a sheath polymer matrix that includes a hydrophobic polymer.

12. The implantable device of claim 11, wherein the hydrophobic polymer includes an ethylene vinyl acetate copolymer.

13. The implantable device of claim 12, wherein the ethylene vinyl acetate copolymer in the sheath has a vinyl acetate monomer content of from about 10 wt. % to about 60 wt. %.

14. The implantable device of claim 12, wherein the vinyl acetate monomer content of the copolymer in the core is greater than the vinyl acetate monomer content of the copolymer in the sheath.

15. The implantable device of claim 11, wherein the sheath polymer matrix constitutes from about 60 wt. % to 100 wt. % of the sheath.

16. The implantable device of claim 11, wherein the sheath is free of a steroidal agent.

17. The implantable device of claim 11, wherein the outer peripheral surface extends between a first longitudinal edge and a second longitudinal edge, wherein at least a portion of the first longitudinal edge, the second longitudinal edge, or both remain uncovered by the sheath.

18. The implantable device of claim 11, wherein the core has a circumferential edge defined between an upper outer peripheral surface and a lower outer peripheral surface, wherein at least a portion of the circumferential edge remains uncovered by the sheath.

19. The implantable device of claim 11, wherein the sheath covers about 50% or more of the outer peripheral surface.

20. The implantable device of claim 11, wherein the sheath covers 100% of the outer peripheral surface.

21. The implantable device of claim 1, wherein the device has a generally circular cross-sectional shape.

22. The implantable device of claim 1, wherein the device is in the form of a cylinder.

23. The implantable device of claim 1, wherein the device has a thickness of from about 0.1 to about 10 millimeters and/or a length of from about 6 to about 250 millimeters.

24. The implantable device of claim 1, wherein the core is formed from a hot melt extrusion process.

25. The implantable device of claim 1, wherein the core is loaded with from about 5 mg to about 30 mg of the steroidal agent.

26. A method for prohibiting and/or treating an inflammatory eye condition in a patient, the method comprising inserting the device of claim 1 in an eye of a patient.

27. The method of claim 26, wherein the device is inserted into the anterior segment of the eye.

28. The method of claim 26, wherein the device is inserted into the posterior segment of the eye.

29. The method of claim 26, wherein the device is inserted into the vitreous humor.
